(19) United States
(12) Patent Application Publication
(10) Pub. No.: US 2024/0169857 A1
(43) Pub. Date: May 23, 2024

(54) PERSONALIZATION OF PROTEINS FOR INGESTION

(71) Applicant: Elo Health, Inc., West Yellowstone, MT (US)

(72) Inventors: Pasha Gurevich, Berkeley, CA (US); Tapio Tolvanen, Cupertino, CA (US); Ari Tulla, San Francisco, CA (US); Jussi Kolehmainen, Helsinki (FI); Zuzana Krejciova-Rajaniemi, Corte Madera, CA (US)

(21) Appl. No.: 18/516,841

(22) Filed: Nov. 21, 2023

Related U.S. Application Data
(60) Provisional application No. 63/427,644, filed on Nov. 23, 2022.

(51) Int. Cl. G09B 19/00; G16B 20/00; G16H 20/60

(52) U.S. Cl. CPC G09B 19/0092; G16B 20/00; G16H 20/60

(57) ABSTRACT

Embodiments can create smart protein, a personalized protein product that is calibrated to a user based on at least one of the three sources of data: a blood biomarker panel, activity (i.e., movement) data, and questionnaire. The smart protein can comprise any set of the following nutrient compositions: protein, carbohydrates, added nutrients, and flavors. The ingredients and the doses of each of the following components of nutrients can be personalized based on the three sources of data. For example, carbohydrate and protein doses of smart protein for one user may be completely different than smart protein of another user based on the three sources of data.

### CROSS-REFERENCES TO RELATED APPLICATIONS

The present application claims priority from and is a non-provisional application of U.S. Provisional Application No. 63/427,644, entitled “Personalization Of Proteins For Ingestion” filed on Nov. 23, 2022, the entire contents of which are herein incorporated by reference for all purposes.

### BACKGROUND

Although there are many protein supplements out in the market, only few protein supplements are personalized. Even the few protein supplements that are personalized do so at a high level of abstraction, i.e., based only on a questionnaire such as gender, age, weight, etc. While these data points yield the ability to create a more personalized product than no data points, they are still far from being truly and fully personalized.

Embodiments of the disclosure address this problem and other problems individually and collectively.

### SUMMARY

One embodiment of the disclosure includes a method. The method can comprise a computer system receiving movement data from one or more user devices of a user, the movement data representative of activity levels of the user over a specified number of days. The movement data can be measured by the one or more user devices. The computing system can then receive user data including height, weight, age, and gender. The computing system can determine, by the computing system, a total active calorie burn using the movement data and an activity score using the total active calorie burn and the user data. The computing system can then determine a carbohydrate dose based on the activity score. The carbohydrate dose can be mixed with a protein dose to obtain a protein-carbohydrate composition for the user.

Another embodiment of the disclosure includes a method by a computing system. The method can comprise the computing system receiving a first assessment including a first measurement of a first biomarker of a user and a second assessment including a second measurement of a second biomarker of the user or a first health goal of the user. The computing system can then query a database using the first assessment and the second assessment to: 1) obtain a plurality of first reference ranges for a first nutrient associated with the first assessment, 2) obtain a plurality of second reference ranges for the first nutrient associated with the second assessment, and 3) obtain one or more additional references ranges for one or more other nutrients. The plurality of first reference ranges can be associated with corresponding first efficacies and corresponding first weights of the first nutrient for the first assessment. The plurality of second reference ranges can be associated with corresponding second efficacy and corresponding second weights of the first nutrient for the second assessment. The computing system can determine a first dosage bound of the first nutrient for the first assessment using the plurality of first reference ranges and the corresponding first weights of the first nutrient for the first assessment, and a second dosage bound of the first nutrient for the second assessment using the plurality of second reference ranges and the corresponding second weights of the first nutrient for the second assessment. The computing system can then generate a dosage vector including a dose value for each of a plurality of nutrient products including the first nutrient and the one or more other nutrients. The computing system can generate a score matrix that includes a nutrient-assessment score for each nutrient for each assessment. The nutrient-assessment score can be dependent on the dose value for the nutrient relative to a dosage bound for the nutrient. The computing system can optimize the dosage vector by optimizing a total score across nutrients and assessments, thereby obtaining an optimal dose for each of the plurality of nutrient products. The total score can be determined using the score matrix and an efficacy for the nutrient for each reference range for each assessment. The computing system can create a combination product that includes a protein product and the optimal dose for each of the plurality of nutrient products; and provide the combination product to the user.

Yet another embodiment of the disclosure includes a method performed by a computing system. The method can comprise the computing system receiving historical movement data from one or more user devices of a user, the historical movement data representative of activity levels of the user over a specified number of days. The historical movement data can be measured by the one or more user devices. The computing system can then determine a historical active calorie burn rate using the historical movement data. The computing system can receive current movement data from one or more user devices of a user, the current movement data representative of an activity of the user and received within a specified time window of the activity occurring. The current movement data can be measured by the one or more user devices. The computing system can then determine a current active calorie burn using the current movement data and an activity-based dose of a nutrient composition comprising a protein dosage based on the historical active calorie burn rate and the current active calorie burn. The computing system, responsive to receiving the current movement data, can send a notification to a user device to the user, the notification including the activity-based dose for the user to ingest within the specified time window.

A better understanding of the nature and advantages of embodiments of the invention may be gained with reference to the following detailed description and accompanying drawings.

### BRIEF DESCRIPTION OF THE DRAWINGS

FIG. 1 shows a flow diagram of different data used to create smart protein according to some embodiments.

FIG. 2 shows a block diagram of smart protein compositions according to some embodiments.

FIG. 3 shows a flow diagram of determining carbohydrate dose according to some embodiments.

FIGS. 4A and 4B show a flow diagram of determining dosing for micronutrients according to some embodiments.

FIG. 5 shows smart protein packaging according to some embodiments.

FIG. 6 shows an exemplary mobile page for a workout according to some embodiments.

FIG. 7 shows a flow diagram of determining activity-based dose according to some embodiments.

FIG. 8 shows exemplary mobile pages for activity history and ingredients of smart protein according to some embodiments.

FIG. 9 shows exemplary notifications according to some embodiments.

FIG. 10 shows a block diagram of an example computer system usable with systems and methods according to embodiments of the present disclosure.

### TERMS

Prior to discussing embodiments of the disclosure, some terms can be described in further detail.

A “user” may include an individual. In some embodiments, a user may be associated with one or more personal accounts and/or mobile devices. The user may also be referred to as a cardholder, account holder, or consumer.

A “computing device” may include any suitable device that can electronically process data. Examples of computing devices include desktop computers, mobile devices or mobile computing devices, television sets, etc.

A “server computer” may include a powerful computer or cluster of computers. For example, the server computer can be a large mainframe, a minicomputer cluster, or a group of servers functioning as a unit. In one example, the server computer may be a database server coupled to a Web server. The server computer may be coupled to a database and may include any hardware, software, other logic, or combination of the preceding for servicing the requests from one or more client computers.

### DETAILED DESCRIPTION

Embodiments can create smart protein, a personalized protein product that is calibrated to a user based on at least one of the three sources of data: a blood biomarker panel, activity (i.e., movement) data, and questionnaire. The smart protein can comprise any set of the following nutrient compositions: protein, carbohydrates, added nutrients, and flavors. The ingredients and the doses of each of the following components of nutrients can be personalized based on the three sources of data. For example, carbohydrate and protein doses of smart protein for one user may be completely different than smart protein of another user based on the three sources of data.

Embodiments can determine a personalized carbohydrate dose for a user. The carbohydrate dose for the user can be determined based on the user's activity score, wherein the activity score is determined using a total active calorie burn and the user's basal metabolic rate (BMR). The total active calorie burn can be determined by the movement data of the user received through one or more user devices of the user. The user's basal metabolic rate can be determined from user data including height, weight, age, and gender received from the questionnaire. The carbohydrate dose for the user can be mixed along with a protein dose to obtain smart protein personalized to the user.

Embodiments can determine dosage bounds for different micronutrients such as vitamin D or zinc according to the assessments in the biomarker data received from a blood test and questionnaire of the user. The biomarker data and the questionnaire can provide with different assessments to improve different conditions (e.g., vitamin D deficiency). The dosage bounds can be determined by obtaining a plurality of reference ranges for different micronutrients, where each reference range among the reference ranges can have efficacy scores that determine how reliable the reference range is supported by clinical evidence. In some embodiment, upon determining the dosage bounds for different micronutrients, a combination product with optimal dose for plurality of nutrient products (e.g., 25 mg of vitamin D pill, 50 mg of vitamin D pill, etc.) can be determined.

Embodiments can determine activity-based dose of the smart protein for a user. The activity-based dose can be determined by using movement data of the user received through one or more user devices of the user. The one or more devices can measure current movement data (e.g., current exercise period) and historical movement data over a period of time (e.g., past 30 days). From these data, a current active calorie burn and historical average active calorie burn can be determined. Based on the current active calorie burn and the historical average active calorie burn, the activity-based dose of the smart protein for the user can be determined.

### I. Personalized Composition for User

Smart protein can be a personalized protein product that is calibrated to a user based on at least one of the three sources of data: a blood biomarker panel, movement data, and questionnaire. The blood biomarker panel can be measured using a blood biomarker test. The movement data can be measured using one or more of user's devices (e.g., Smart watch). The questionnaire can be user data taken from different questions asked to a user.

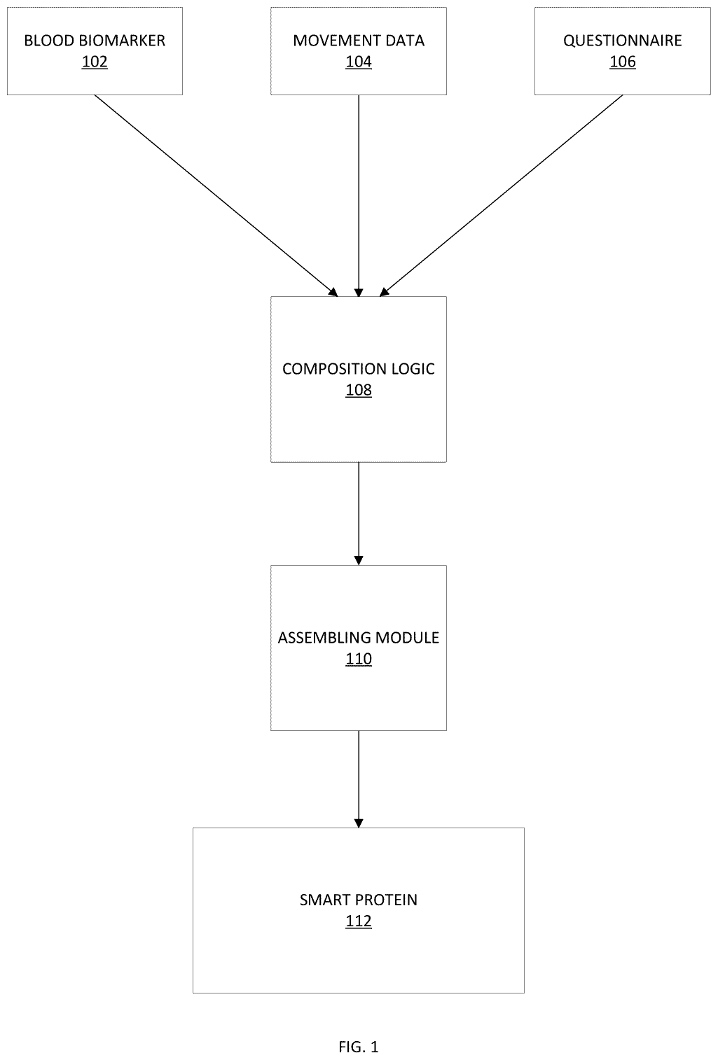

FIG. 1 shows a flow diagram of different data used to create smart protein 112 according to some embodiments. At least one of blood biomarker 102, a movement data 104, and a questionnaire 106 of a user can be used to create the smart protein 112 for the user. Based on the data, the smart protein 112 can use a composition logic 108 to create smart protein. The composition logic 108 can determine the types and doses of ingredient based on the data. Upon determining different types and doses of ingredients, an assembling module 110 can assemble the ingredients and doses according to the composition logic 108 and determine the smart protein 112.

### A. Blood Biomarker

The blood biomarker 102 can be obtained by the user completing blood biomarker testing. The blood biomarker test can analyze key micronutrient levels, the body's circulatory capacity, a lipid panel, and measures of inflammation. The biomarker testing can be carried out by using a bioluminescence technique. Example lab analyzers using the bioluminescence technique can include Beckman Coulter Au680 chemistry analyzer, Beckman coulter DXI 800 chemistry analyzer, etc. Measured biomarkers can include: total cholesterol, low-density lipoprotein (LDL), high-density lipoprotein (HDL), triglycerides, Hemoglobin A1c, vitamin D, vitamin B12, ferritin, homocysteine, etc. The test can be performed on the user by using a finger-prick on an advanced diagnosis (ADx) collection device. The dry-blood sample of the user can be then shipped to a laboratory for analysis.

The micronutrient levels can help analyze different micronutrients the user needs. For example, if the blood biomarker 102 comes with data that the user is low on vitamin D, then the composition logic 108 can determine ingredients and doses that can help the user increase the vitamin D level in the smart protein 112.

### B. Movement Data

The movement data 104 can be obtained from the one or more user devices (e.g., smart watch). The movement data 104 can provide with historical movement data over a period of time (e.g., past 30 days). The movement data 104 can also provide with real-time current movement data including the type of activity, intensity, and duration of the current movement (e.g., a workout session). The historical movement data can be used to determine historical active calorie burn during the period of time. The historical active calorie burn can be divided by the period of time to determine a historical active calorie burn rate. For example, if the user burned 30000 calories over a span of 30 days doing different activities, then the historical active calorie burn rate can be 100 calories/day. The real-time current movement data can be used to determine a current active calorie burn.

The composition logic 108 can use the historical active calorie burn rate to determine carbohydrate dose composition of the smart protein. The composition logic 108 can determine a quantity ratio of carbohydrate to protein in the smart protein 112. For example, if the user has high historical active calorie burn rate, then the composition logic 108 can assign the user with higher ratio of carbohydrate than protein such as increasing dextrin carbohydrates that replenish glycogen stores without spiking blood sugar.

### C. Questionnaire

The questionnaire 106 can be user data obtained from the user filling out different questions via online. The questionnaire 106 can comprise user's health goals, dietary restrictions (e.g., allergies), gender, age, weight, activity type, activity level, etc. Similar to other types of data (e.g., blood biomarker 102 or movement data 104), the composition logic 108 can determine ingredient and doses based on the user's answers to the questionnaire 106. For example, if the user is sensitive to lactose, then the composition logic 108 can assign the protein composition of the smart protein to be that of pea protein. If the user is not sensitive to lactose, then whey protein can be used. The user's questionnaire can also be used to determine the user's basal metabolic rate (BMR). Additionally, the questionnaire 106 (e.g., answers to goals) can be used to determine dosages of micronutrients.

### D. Composition and Assembling

The composition logic 108 can use blood biomarker 102, movement data 104, and the questionnaire 106 to determine different ingredients that can satisfy protein, carbohydrates, micronutrients, and flavor of the smart protein, and appropriate doses personalized for the user. Certain nutrients can require combination of different data to determine the ingredient and doses. For example, when determining the carbohydrate dose, the composition logic 108 can use the user's basal metabolic rate (BMR) from the questionnaire 106 and historical active calorie burn rate from the movement data 104.

The composition logic 108 can have a plurality of algorithms in determining the types and doses of ingredients. The protein dose can be determined based on the data from questionnaire 106 including the user's weight, the type of protein, etc. The carbohydrate dose can be determined based on the quantity ratio of carbohydrate to protein and the protein dose. The quantity ratio can be determined based on the user's BMR, historical active calorie burn rate, etc. The micronutrients and their dosages can be determined by different assessments using the blood biomarker, efficacy levels, and the user's goal according to the questionnaire data.

The assembling module 110 can receive instructions from the composition logic 108 of different ingredients and doses for the smart protein 112. The assembling module 110 can assemble the different ingredients (e.g., protein, carbohydrates, micronutrients, and flavor) according to the instructions from the composition logic 108. The assembling module 110 can take stocks of the different ingredients and take measurement of appropriate dosages on scale based on final volume of the bag and the instruction from the composition logic 108. The assembling module 110 can then add the appropriate dosage of the ingredient to a mixer machine, which can mix the ingredients. The different dosages of different ingredients can be then mixed in the mixer machine until they become homogenous (i.e., reach homogeneity).

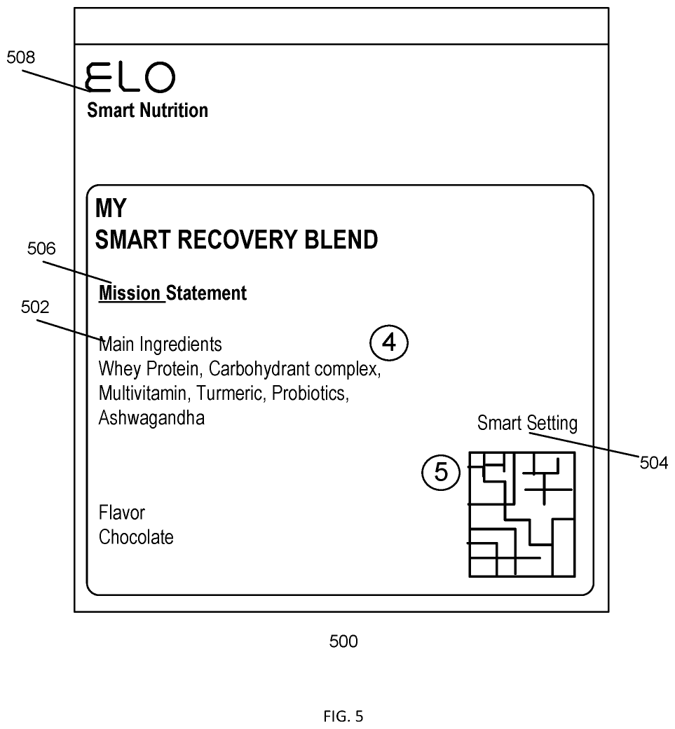

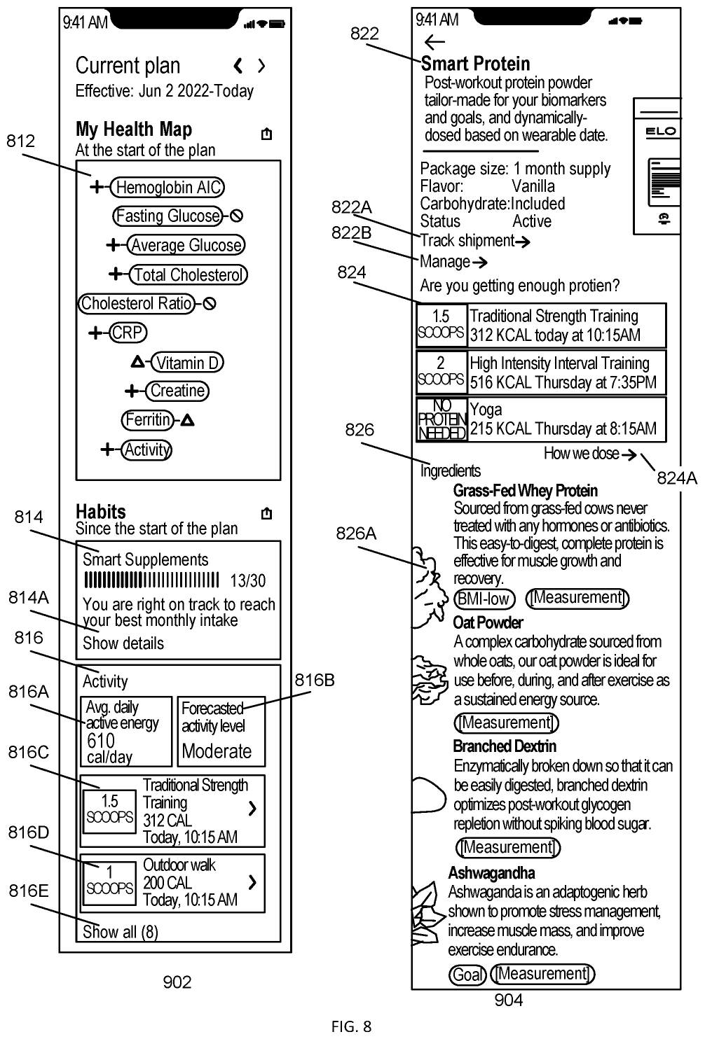

Once the ingredients are well mixed and homogenous, the ingredients can then be filled into a bag to create the smart protein 112. The smart protein can be a composition of at least one of the ingredients (e.g., protein, carbohydrate, micronutrients, and flavor). For example, a protein dose and a carbohydrate dose can be added to the mixer machine. The protein dose and the carbohydrate dose can then be mixed in the mixer machine until they become homogenous to determine the smart protein with protein-carbohydrate composition. The assembling module 110 can additionally provide a general description of the types and doses of the ingredients on a package of the smart protein. Descriptions of the package can be further explained in FIG. 5. The assembling module 110 can further write detailed explanation on algorithms on how the type and doses of the ingredients have been determined in a separate user application further described in FIG. 8.

### II. Smart Protein Ingredients

Smart protein can comprise different nutrients that can help with post-workout recovery, maintain energy levels, and prevent muscle soreness after activity. Different doses of ingredients for the nutrients can be mixed to create smart protein. The doses can be determined based on a user's activity level, user's goal, dietary restrictions, etc. New smart protein can be determined for every period of time (e.g., 30 days) by using new movement data, blood biomarker data, and/or questionnaire 106 collected during the most recent period of time.

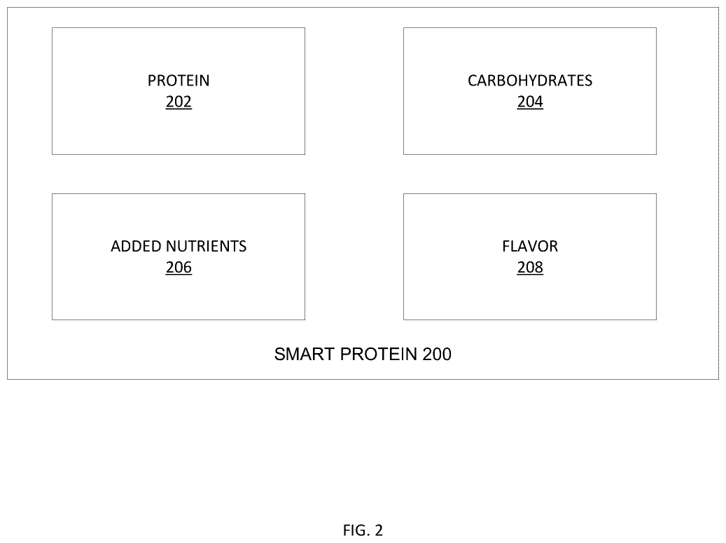

FIG. 2 can show a block diagram of smart protein compositions according to some embodiments. The smart protein can comprise protein 202, carbohydrates 204, added nutrients 206, and flavor 208. Different doses of protein 202, carbohydrates 204, added nutrients 206, and flavor 208 based on the data (e.g., blood biomarker, activity data, questionnaire) from FIG. 1 can be mixed to create smart protein 200.

### A. Protein

The protein 202 can be of different types such as whey protein, pea protein, etc. The protein 202 type can be determined by any dietary restrictions and/or preferences specified by the user. This information can be derived from the questionnaire data. The protein dosing can be body weight corrected and calculated to achieve at least 3 grams of amino acid leucine per serving (e.g., per day), where the amino acid leucine is a primary activator of the mammalian target of rapamycin (mTOR) biochemical pathway associated with muscle protein synthesis. Protein dosing can be restricted to not exceed the level at which an individual may be determined to no longer benefit from additional intake (e.g., ˜40 g per serving).

Different protein type can have different amino acid leucine composition, and this can be factored into each individual recommendation. For example, pea protein and whey protein can have slightly different amino acid leucine compositions and would be factored differently. Different protein type can be selected based on the user's dietary restrictions. For example, if a user has a sensitivity to lactose or follows a restrictive diet, a pea protein can be used. Otherwise, whey protein can be used.

Protein dose can be determined based on an algorithm. Body weight, a fixed ratio (e.g., ideal ratio determined to give best efficacy), minimum protein dose, and a maximum protein dose can be used to determine an appropriate protein dose for a user. The following algorithm can be used to determine protein dose.

```
body_weight_dose = body_weight_kg * 0.4 g/kg/serving
minimum_protein_dose = 3 g / protein_type_leucine_percentage
Maximum_protein_dose = 40g / serving
if body_weight_dose < minimum_protein_dose:
  final_protein_dose = minimum_protein_dose
else:
  if body_weight_dose < maximum_protein_dose:
    final_protein_dose = body_weight_dose
  else:
    final_protein_dose = maximum_protein_dose
```

where body_weight_kg is a variable name for body weight in kilograms, body_weight_dose is a variable name for an amount of dose determined using the body_weight_kg and the fixed ratio (e.g., 0.4 g/kg/serving), minimum_protein_dose can be a variable name for minimum protein dose, maximum_protein_dose can be a variable name for maximum protein dose, and protein_type_leucine_percentage can be the amino acid leucine percentage that is unique to different protein type, and final_protein_dose can be a variable name for final protein dose for the smart protein 200.

### B. Carbohydrates

The carbohydrates 204 can comprise a plurality of carbohydrates such as oat starch, dextrin, etc. Different carbohydrate types can provide different effects. For example, dextrin carbohydrate can be a fast-digesting carbohydrate that can replenish glycogen stores without severely spiking blood sugar. In comparison, oat starch can be a slow-digesting carbohydrate with slower rate of replenishing glycogen stores but providing energy over an extended period of time.

The carbohydrate dose of smart protein for a user is proportional to the user's historic activity level over a period of time (i.e., historical active movement data). For example, if the user has high activity level resulting in high active calorie burn, then high carbohydrate doses can be used in making the smart protein 200. The carbohydrate is important for building muscle, as the body looks to glycogen for energy after a workout (i.e., an activity). Without enough doses of glycogen, the body can break down muscle tissue for energy, which can result in muscle loss. Therefore, fast-digesting carbohydrates that can replenish glycogen stores quickly can be used to spike the user's glycogen after the workout, leading to increase in fast-digesting carbohydrate composition in the smart protein such as Dextrin with increase in the user's activity level.

The carbohydrate dose of smart protein can be determined by using the user's historical active movement data and the user's BMR. The user's historical active movement data can be measured using one or more user devices. The one or more user devices can also determine historical active calorie burn from the historical active movement data. The user's BMR can be determined by using the questionnaire data such as the user's gender, age, height, weight, etc. The user's BMR and the historical active calorie burn can then be used in an algorithm to determine carbohydrate dosages personalized for the user. Details of the algorithm can be found in later section.

### C. Added Nutrients

The added nutrients 206, or micronutrients, can be functional nutrient boosts such as multivitamin, probiotics, turmeric, etc. The embodiment can take the user's blood sample and perform blood biomarker test to see status of different nutrients and micronutrients in the user's body. The biomarker data from the biomarker test can show status of various biomarkers (e.g., vitamin A, vitamin D, cholesterol, etc.) in the user's body. The status can be divided into five different categories in terms of how deficient the user is of the specific biomarker. For example, the status can be divided into vey very low, low, optimal, elevated, and high. The status can indicate the severity of how much the user needs the biomarker, and this can play into effect when determining what micronutrients, and the dosages of the micronutrients, are added into the smart protein 200.

The added nutrients 206 can also be based on the user's questionnaire. For example, the added nutrients 206 based on increasing the immunity of the user can be very different than the added nutrients 206 based on losing weight.

In some embodiments, the added nutrients 206 can be provided on the side as supplements (e.g., pills) instead of being part of the smart protein 200. The added nutrients 206 can be generated as additional supplements that the user can take alongside the smart protein 200. For example, the added nutrients 206 can be pills that the user can take along with the smart protein 200. Similar to the smart protein 200, the user can determine new set of added nutrients 206 at every period of time (e.g., 30 days).

### D. Flavor

To enhance the taste of the smart protein 200, different flavor 208 can be added. Examples of flavor 208 can be chocolate, vanilla, plain, etc. The flavor 208 can be chosen by the user. The flavor 208 can help add extra taste to the smart protein 200 such that the user can enjoy taking the smart protein 200 better.

In some embodiments, the added nutrients may be not mixed with other nutrients in the smart protein 200. Instead, the added nutrients can be supplied as extra supplements besides the smart protein 200 to enhance the flavor of the smart protein 200.

### III. Carbohydrate Mixing

The carbohydrates can be of different types such as oat starch, dextrin, etc. Different carbohydrate types can have different effects. A combination of different types of carbohydrate can be used to comprise the carbohydrates composition of the smart protein. For example, the carbohydrate can comprise a mixture of oat starch and dextrin.

The carbohydrate ingredient and doses can be determined using questionnaire data and movement data representative of activity levels of the user over a specified number of days. The questionnaire data can be used to determine a user's BMR, as the user's BMR uses data such as the user's gender, age, height, and weight to determine the BMR. The movement data can be used to determine a total active calorie burn over the specified number of days.

### A. Determining Carbohydrate Mixing

The carbohydrate dose can be anchored to protein dose with a quantity ratio determined by using an activity score. The quantity ratio can be ratio between protein to carbohydrates. The activity score can be determined by using a total active calorie burn over a span of time (e.g., last 30 days) determined by using movement data and the user's basal metabolic rate (BMR). The total active calorie burn can be determined by aggregating calorie burns of different activities over the specified span of time (i.e., number of days). For example, a total sum of the calorie burn for each day can be determined. The sum can be a weighted sum. The movement data can represent activity levels of the user over a specified number of days and can be measured using the one or more user devices (e.g., smart watch). The user's basal metabolic rate (BMR) can be determined from questionnaire data including user's gender, age, height, and weight. The user's BMR can estimate the calories burned by the user to maintain vital life functions (e.g., heartbeat). The BMR can be summed over the span of time.

The quantity ratio for carbohydrates can increase with increased activity level from the user. An example table that shows different activity scores with different quantity ratios can be shown below in Table 1.

TABLE 1

| | No | Low | Med | High |
|---|---|---|---|---|
| | Carb | Carb | Carb | Carb |
| Protein | 100% | 85% | 65% | 50% |
| Carb | 0% | 15% | 35% | 50% |
| | | (5% Oat Starch + | (5% Oat Starch + | (5% Oat Starch + |
| | | 10% Dextrin) | 30% Dextrin) | 45% Dextrin) |

where the activity score of “No Carb” can represent the quantity ratio of little to no activity level, the activity score of “Low Carb” can represent the quantity ratio of little to medium activity level, the activity score of “Med Carb” can represent the quantity ratio of medium to high activity level, and the activity score of “High Carb” can represent the quantity ratio of high activity level. By using the activity score, carbohydrate dose can be determined. Example carbohydrate dosage of dextrin (HBCD) carbohydrates and oat starch carbohydrates in comparison to protein in a bag of 500 grams can be shown below in Table 2.

TABLE 2

| Per ~500 g Bag | Protein (g) | HBCD (g) | Oat Starch (g) |
|---|---|---|---|
| No Carb | 500 | — | — |
| Low Carb | 425 | 50 | 25 |
| Medium Carb | 325 | 150 | 25 |
| High Carb | 250 | 225 | 25 |

Carbohydrate dose can be determined using the following algorithm. Different percentages of the user's BMR and the total active calorie burn can be used to determine the activity score. The following algorithm can be used to determine the activity score.

```
If total_active_calorie_burn <= 5% of user_bmr OR user_preference
  No_carb_bucket
Else if total_active_calorie_burn > 5% of user_bmr and total_active_calorie_burn <= 33% of
user_bmr
  Low_carb_bucket
Else if total_active_calorie_burn > 33% of user_bmr and total_active_calorie_burn <= 66% of
user_bmr
  Med_carb_bucket
Else if total_active_calorie_burn > 66% of user_bmr
  High_carb_bucket
```

where total_active_calorie_burn is a variable name for total active calorie burn over the span of time, a user_bmr can be a variable name for the user's BMR, user_preference can be a variable name for user selected limit (i.e., user preference rate), no_car_bucket can be a variable name for an activity score of “No Carb”, low_car_bucket can be a variable name for an activity score of “Low Carb”, med_carb_bucket can be a variable name for an activity score of “Med Carb”, and high_carb_bucket can be a variable name for an activity score of “High Carb”. In some embodiments, in determining the activity score of “No Carb”, the user can set its own personalized limit (i.e., user preference rate) of more than 5% of user's BMR.

### B. Example

An illustrative example of determining the smart protein 200 ingredients and its doses of nutrients can be shown below. Following exemplary data of protein in FIG. 2, which can be based off of at least one of user questionnaire, biomarker, or activity data, can be used as an example.
Scoop size: ˜32 g
Gender: Male
Age: 34
Height: 5′6″
Weight: 150 lb (68.0389 kg)
Daily BMR (totaled over previous 30 days): 32,826
Active Calorie Burn (totaled over previous 30 days: 12,209
Carbohydrate bucket: Medium
Dairy/lactose intolerance: None
Vegan diet preference: Not followed
Dairy intake: Deficient
Probiotic intake: Deficient
Goal: Performance
Blood biomarkers indicate moderate inflammation

Since the active calorie burn is greater than 33% of the user's BMR but smaller than 66% of the user's BMR, the activity score can be medium, with the quantity ratio being 5% oat starch, 30% dextrin, and 65% protein. Therefore, this can lead to the following specification of the smart protein 200.

One Bag of Product (˜500 g)
Protein: Whey Protein (65%)
Carbohydrate Complex: Branched Dextrin (30%)+Oat Starch (5%)

Assuming that one scoop of smart protein has 32 grams of smart protein, each scoop's percentage of ingredients can be calculated using the quantity ratio. The specification for the scoop of 32 grams can be of the following.

```
Per Scoop
32 g scoop = {
  Oat_starch (5%) = 1.6 g
  Branched_dextrin (30%) = 9.6 g
  Whey_protein (65%) = 20.8 g
}
```

Since the amount of protein that is contained in a scoop can be calculated, the number of scoops to achieve ideal clinical efficient dose of 3 grams of leucine protein can be calculated according to protein dose algorithm in FIG. 2. Since whey protein, which is the protein used in the example, consists of 10.8% of amino acid leucine percentage, then the minimum protein dose can be 3 g/10.8%=27.78 grams. Since each scoop has 20.8 g of protein dose, to meet the minimum protein dose of 27.78 grams, there can be 27.78 g/(20.8 g/scoop)=1.3 scoops of the smart protein to achieve minimum protein dose. Since the user's body weight dose 68.0389 kg*0.4 g=27.22 grams is smaller than the minimum protein dose of 27.78, the 1.3 scoops, or the 27.78 g of protein dose, can be the dose that the user can take.

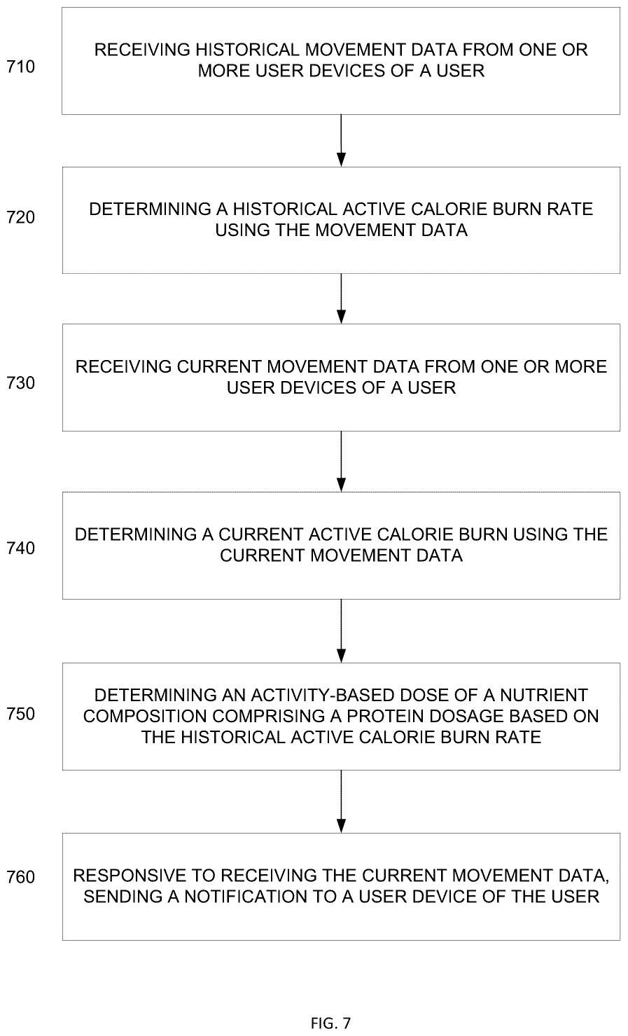

This 1.3 scoops can be known as a reference dose and can later be used to calculate activity-based dose of smart protein determined in FIG. 7. As seen through the example calculation, the reference dose can be dependent on ingredient, protein composition in smart protein, and dose (scoop) size, The reference dose can be different for users with different ingredients, activity score, etc.

### C. Flowchart

Methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps.

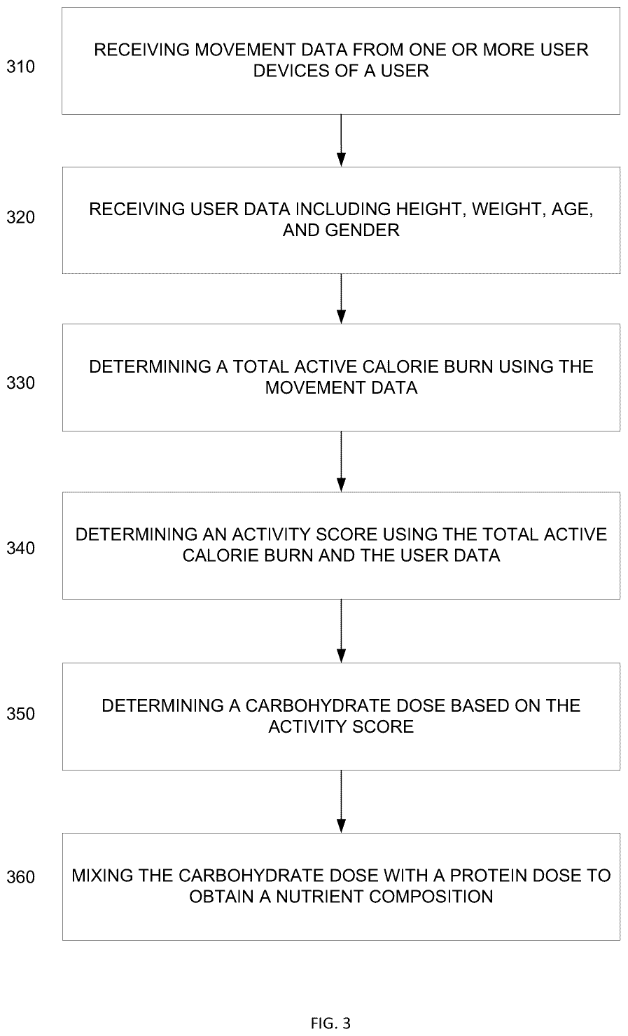

FIG. 3 shows a flow chart for determining a carbohydrate dose according to some embodiments. The method 300 shows a computer system receiving movement data and user data to determine an activity score that can be used to determine the carbohydrate dose for a user.

In step 310, the computing system can receive movement data from one or more user devices of a user. The movement data can be representative of activity levels of the user over a specified number of days (e.g., 30 days). For example, the movement data can track different types of activities such as running, playing basketball, yoga, etc. that the user did over the specified number of days. The movement data can also be known as historical active movement data in the method 300. The movement data can be measured by the one or more user devices (e.g., Smart Watch). The movement data can be tracked and stored by the one or more user devices by using an application. Different activity details such as time, heart rate, amount of distance traveled, etc. can be measured by the one or more user devices. These activities can be collected over the specified number of days to determine the movement data.

In step 320, the computing system can receive user data. The user data can be questionnaire data with answers to different health questions including height, weight, age, gender, etc. The user data can be provided by the user to the computing system. The user data can be used to determine the user's BMR. The user's BMR can be dependent on the height, weight, age, and gender of the user. The BMR calculation for a man and a woman can be

BMRₘₐₙ=10*W+6.25*H−5A+5

BMR_{woman}=10W+6.25H−5A−161

Where W is weight in kilograms, H is body height in centimeters, and A is age.

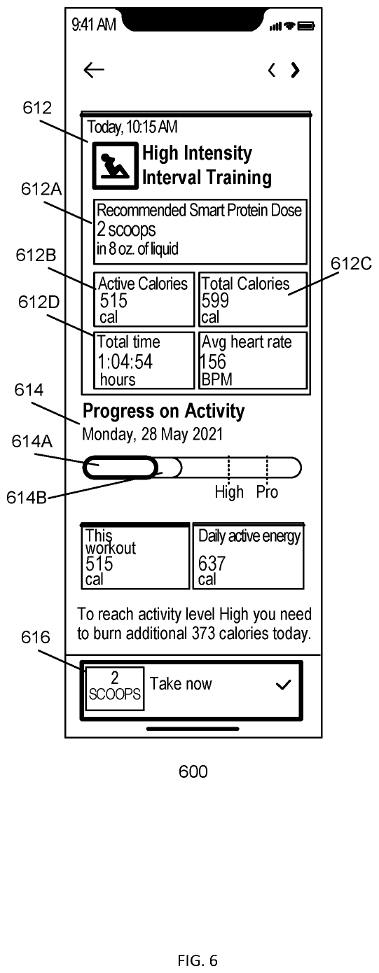

In step 330, the computing system can determine a total active calorie burn using the movement data. The total active calorie burn (i.e., historical active calorie burn) can be total calorie burn of the user over the period of time (e.g., 30 days) by doing different activities. Different activities can lead to different active calorie burns. The total active calorie burn can be determined by aggregating calorie burns of different activities over the period of time (i.e., specified number of days). The active calorie burn can also be dependent on a user. For example, running for an hour can lead to a lot higher active calorie burn than doing YOGA for an hour. In some embodiments, the one or more user devices can, along with measuring the movement data, determine the calorie burn of each activity. For example, in FIG. 6, along with collecting the activity of the user, the total active calories burned can be shown. The active calorie burns of the different activities can then be added to determine the total active calorie burn.

In step 340, the computing system can determine an activity score using the total active calorie burn and the user data. The activity score can be categorized into four different scores: No Carb, Low Carb, Med Carb, and High Carb. The BMR determined using the user data can be compared with the total active calorie burn to determine the activity score. The higher the total active calorie burn is, the more likely the activity score will fall into higher activity score (e.g., High carb). In some embodiments, instead of using the BMR, the user can set its own user preference to determine what the boundary of No Carb can be.

In step 350, the computing system can determine a carbohydrate dose based on the activity score. The activity score can determine what the protein to carbohydrate ratio (i.e., quantity ratio) will be. For example, Low Carb score can lead the protein to carbohydrate ratio to be 85 percent protein and 15 percent carbohydrate. The activity score can also determine what the carbohydrate composition should be. For example, for Low Carb score, the carbohydrate composition can be 5 percent oat starch carbohydrate and 10 percent dextrin carbohydrate. As the protein dose can be determined according to the algorithm in FIG. 2, upon finding the protein to carbohydrate ratio, the carbohydrate dose can be determined by using the protein to carbohydrate ratio to the protein dose. For example, if the activity score is Low Carb (85% protein and 15% carb) and the protein dose is 85 grams, then the carbohydrate dose can be 15 grams. The carbohydrate dose can specify a quantity of each of a plurality of carbohydrates. For example, the carbohydrate dose can be 30% carbohydrate composed of 5% oat starch and 25% Dextrin.

In step 360, the computing system can mix the carbohydrate dose with a protein dose to obtain a nutrient composition of the smart protein for the user. The smart protein can comprise other nutrients such as micronutrients that is determined by using blood biomarker data. The smart protein can also add ingredients that can enhance the flavor.

### IV. Nutrient Additions

Additional nutrients (i.e., micronutrients) can provide other supplementary micronutrients such as vitamins, probiotics, turmeric, etc. to the smart protein. The additional nutrients personalized for a user can be determined by taking a biomarker test. The biomarker test can provide with biomarker data that show status of different biomarkers of the user. Based on the status of different biomarkers, the embodiment can determine additional nutrients. For example, if the biomarker data shows that the user is lacking in vitamin D, the additional nutrients can comprise vitamin D supplements. In addition to the biomarker data, questionnaire data specifying the user's goal can also be included to determine the additional nutrients.

### A. Determining Nutrient Additions

Additional nutrients can be determined by using an algorithm that determines the ingredients and the dosages of the additional nutrients. The algorithm can first collect biomarker data and questionnaire data. The algorithm can select different biomarkers that the user is deficient in. For example, if the vitamin D status is very low, then the algorithm can determine the added nutrients that boosts the vitamin D level. If the user's goal is to increase its immunity level, then nutrients such as vitamin D or zinc can be used to help with immunity.

The algorithm can target different dosing of added nutrients to target all the assessments. For example, if the first assessment is vitamin D deficiency and the second assessment is immunity, the algorithm can work to figure out the dose for the deficiency and the immunity, and then based on the two outcomes can work to find the lowest dose possible to target all the assessments, to be efficacious towards those assessments.

The computing system can receive assessments of different measurements of biomarkers upon collecting biomarker data and questionnaire data. The assessments can identify user's goal stated in the questionnaire data or nutrient deficiencies identified in the biomarker data. For example, the computing system can receive a first assessment including a first measurement of a first biomarker (e.g., Vitamin D), and the computing system can receive a second assessment including a second measurement of a second biomarker or a first health goal of the user (e.g., Immunity). Different assessments can lead to different nutrients recommendations. For example, vitamin D deficiency assessment can recommend doses for vitamin D while goal immunity assessment can recommend doses for both vitamin D and zinc.

Based on the assessments, the computing system can obtain a plurality of first reference ranges for a first nutrient associated with the first assessment. The reference ranges can be ranges of doses of the first nutrient recommended to the user to improve the condition of the assessment. The reference ranges can be determined using different sources such as scientific literatures, research papers, etc. Each of the plurality of first refence ranges can be associated with corresponding first source identifier, first type of source, and a first efficacy. The first source identifier can be a unique identifier that locates a source that was used to determine the first reference range. The first type of source can identify the category of the source (e.g., meta-analysis, systematic review, randomized control trial (RCT), etc.). The efficacy can be a numerical value indicating effectiveness of a given nutrient or nutrient doses in improving or positively affecting the condition of the assessment based on the evidence from human clinical trials and peer-reviewed scientific literature. The high efficacy value can indicate there is high level of reliable clinical evidence supporting the reference range. The efficacy level can be influenced by different type of sources, as the type of study can determine the level of proof of efficacy. For example, a meta-analysis covering 50 different studies offers a greater proof of efficacy than does a single random control trial (RCT).

The efficacy rating can be divided into several different scores: 3 indicating effective, 2 indicating likely effective, 1 indicating possibly effective, 0.5 indicating insufficient reliable evidence to rate to date, and none indicating no reliable evidence. The higher the efficacy rating, the stronger the clinical evidence supporting the effectiveness of a given nutrient in improving or positively affecting the condition given in the assessment (e.g., vitamin D deficiency).

The efficacy rating of 3 can indicate that the nutrient has a very high level of reliable clinical evidence supporting its use for a specific indication. The evidence is consistent with or equivalent to passing a rigorous approval process such as the ones given by a national agency (e.g., Food and Drug Administration (FDA)). The evidence can be from multiple (2+) randomized clinical trials or meta-analysis including several hundred to several thousand patients. The studies where the evidence can have a low risk of bias and high level of validity, and the evidence consistently shows positive outcomes without valid evidence to the contrary. Additionally, it has a strong p value less than 0.001 in indicating that the nutrient is effective.

The efficacy rating of 2 can indicate that the nutrient has a very high level of reliable clinical evidence supporting its use for a specific indication. The evidence can be from multiple (2+) randomized clinical trials or meta-analysis including several hundred patients. The studies where the evidence can have a low risk of bias and high level of validity, and the evidence consistently shows positive outcomes without valid evidence to the contrary. Additionally, it has a strong p value less than 0.001 in indicating that the nutrient is effective. The efficacy rating 2 may not be as strong as 3 in the sense that it does not need to pass a review by a national agency nor need to have the evidence from several thousands of patients.

The efficacy rating of 1 can indicate that the nutrient has some reliable clinical evidence supporting its use for a specific indication. However, the evidence can be limited by quantity, quality, or contradictory findings. The evidence can be from one or more randomized clinical trials, meta-analysis, two or more population-based studies, or epidemiological studies. The studies can have low to moderate risk of bias and moderate to high level of validity by meeting or partially meeting assessment criteria. The evidence can show positive outcomes for a given indication without substantial valid evidence to the contrary. There may be some contrary evidence, but valid positive evidence outweighs contrary evidence. Additionally, it has a p value greater than 0.001 but less than 0.005 in indicating that the nutrient is somewhat effective. The criteria for efficacy rating of 1 is lower than both the efficacy ratings 2 and 3.

The efficacy rating of 0.5 can indicate that there is insufficient reliable evidence to rate to date. There is some, but not enough reliable scientific evidence to provide an efficacy rating. The efficacy ration of blank can indicate that there is no efficacy or insufficient reliable evidence to rate.

As an illustrative example, if the first assessment is Vitamin D deficiency with the first nutrient being vitamin D, the plurality of first reference ranges for the vitamin D can be 25 mg (PubMed ID (PMID) 23168298, meta-analysis, efficacy 3) and 50-125 mg (PMID 31114460, systematic review, efficacy 2). The PubMed ID can be a source identifier referencing a unique clinical study published in PubMed, a digital repository of published clinical and scientific literature, maintained by the National National Library of Medicine. The first reference range 25 mg can have a first source identifier PMID 23168298, a first type of source meta-analysis, and a efficacy 3. The first reference range 50-125 mg can have a first source identifier PMID 31114460, a first type of source review, and the efficacy 2. Different types of sources can be associated with different weights, with higher weights assigned to types of sources with stronger evidence to support the dosages. For example, the source meta can have a weight of 30 while the source review can have a weight of 15. The plurality of first reference ranges can be associated with first weights according to the first types of sources. Other source weights can include a source random control trial (RCT) being 1, a source guideline being 0.75, etc.

The computing system can obtain a plurality of second reference ranges for the first nutrient associated with the second assessment. Each of the plurality of second reference ranges can be associated with corresponding second source identifier, second type of source, and second efficacy. The plurality of second reference ranges can be associated with second weights according to the second types of sources. For example, if the second assessment is Goal Immunity with the first nutrient being vitamin D, the plurality of second reference ranges for the vitamin D can be 125-500 mg (PMID 32334392, systematic review, efficacy 1) and 20-50 mg (PMID 28202713, meta-analysis, efficacy 2). The computing system can obtain one or more additional references ranges for one or more other nutrients. For example, the assessment of Goal Immunity can obtain a plurality of third reference range for a second nutrient zinc. Each of the plurality of third reference ranges can be associated with corresponding third source identifier, third type of source, and third efficacy. The plurality of third reference range can be 50-80 mg (PMID 32992693, meta-analysis, efficacy 1) and 75-100 mg (PMID 28515951, RCT, efficacy 1). The plurality of third reference ranges can be associated with third weights according to the third types of sources.

The computing system can determine a first dosage bound for the first nutrient of the first assessment. The first dosage bound can be dosage recommendation given for the first nutrient of the first assessment accounting for all the plurality of the first reference ranges and the corresponding first weights. A first lower dosage bound of the first dosage bound can be determined using lower bounds of the plurality of the first reference ranges and the corresponding first weights. A first upper dosage bound of the first dosage bound can be determined using upper bounds of the plurality of the first reference ranges and the corresponding first weights.

The first lower dosage bound can be determined using lower bounds of the plurality of the first reference ranges and the corresponding first weights. Each of the lower bound of the plurality of first reference ranges can be multiplied with the corresponding first weights to determine first weighted lower bounds of the plurality of first reference ranges. The first weighted lower bounds can then be summed together and be divided by the summation of first weights to determine the first lower dosage bound. For example, the vitamin D nutrient for the assessment vitamin D deficiency can have the plurality of first reference ranges of 25 mg (PMID 23168298, meta-analysis, efficacy 3), where the 25 mg is both the lower and the upper dosage bound, with the weight of 30 and 50-125 mg (PMID 31114460, systematic review, efficacy 2) with the weight of 15. The first weighted lower bounds of the plurality of first reference ranges for vitamin D deficiency can be 25*30=750 and 50*15=750. The first weighted lower bounds of the plurality of first reference ranges 750 and 750 can then be summed together 750+750=1500, and be divided by the summation of first weights 30+15=45 to determine the first lower dosage bound 7500/45=33.33.

The first upper dosage bound of the first dosage bound can be determined using upper bounds of the plurality of the first reference ranges and the corresponding first weights. Each of the upper bound of the plurality of first reference ranges can be multiplied with the corresponding first weights to determine first weighted upper bounds of the plurality of first reference ranges. The first weighted upper bounds can then be summed together and be divided by the summation of first weights to determine the first upper dosage bound. For example, the vitamin D nutrient for the assessment vitamin D deficiency can have the plurality of first reference ranges of 25 mg (PMID 23168298, meta-analysis, efficacy 3), where the 25 mg is both the lower and the upper dosage bound, with the weight of 30 and 50-125 mg (PMID 31114460, review, efficacy 2) with the weight of 15. The first weighted upper bounds of the plurality of first reference ranges for vitamin D deficiency can be 25*30=750 and 125*15=1875. The first weighted upper bounds of the plurality of first reference ranges 750 and 1875 can then be summed together 750+1875=2625, and be divided by the summation of first weights 30+15=45 to determine the first upper dosage bound 1500/45=58.33.

The computing system can determine a second dosage bound for the first nutrient of the first assessment. The second dosage bound can be dosage recommendation given for the first nutrient of the second assessment accounting the all the plurality of the second reference ranges and the corresponding second weights. A second lower dosage bound of the second dosage bound can be determined using lower bounds of the plurality of the second reference ranges and the corresponding second weights. A second upper dosage bound of the second dosage bound can be determined using upper bounds of the plurality of the second reference ranges and the corresponding second weights.

The second lower dosage bound can be determined using lower bounds of the plurality of the second reference ranges and the corresponding second weights. Each of the lower bound of the plurality of second reference ranges can be multiplied with the corresponding second weights to determine second weighted lower bounds of the plurality of second reference ranges. The second weighted lower bounds can then be summed together and be divided by the summation of second weights to determine the second lower dosage bound. For example, the vitamin D nutrient for the assessment Goal Immunity can have the plurality of second reference ranges of 125-500 mg (PMID 32334392, systematic review, efficacy 1) with the weight of 15 and 20-50 mg (PMID 28202713, meta-analysis, efficacy 2) with the weight of 30. The second weighted lower bounds of the plurality of second reference ranges for vitamin D deficiency can be 125*15=1875 and 20*30=600. The second weighted lower bounds of the plurality of second reference ranges 1875 and 600 can then be summed together 1875+600=2475, and be divided by the summation of second weights 15+30=45 to determine the second lower dosage bound 2475/45=55.

The second upper dosage bound can be determined using upper bounds of the plurality of the second reference ranges and the corresponding second weights. Each of the upper bound of the plurality of second reference ranges can be multiplied with the corresponding second weights to determine second weighted upper bounds of the plurality of second reference ranges. The second weighted upper bounds can then be summed together and be divided by the summation of second weights to determine the second upper dosage bound. For example, the vitamin D nutrient for the assessment Immunity can have the plurality of second reference ranges of 125-500 mg (PMID 32334392, review, efficacy 1) with the weight of 15 and 20-50 mg (PMID 28202713, meta, efficacy 2) with the weight of 30. The second weighted upper bounds of the plurality of second reference ranges for vitamin D deficiency can be 500*15=7500 and 50*30=1500. The second weighted upper bounds of the plurality of second reference ranges 7500 and 1500 can then be summed together 7500+1500=10000, and be divided by the summation of second weights 15+30=45 to determine the second upper dosage bound 10000/45=222.22.

The computing system can determine dosage bounds for other nutrients in the assessments using the one or more additional reference ranges. The dosage bounds for other nutrients can be determined using the method similar to determining dosage bounds of the first nutrient of the first and second assessments. For example, zinc nutrient for the assessment Immunity can have the plurality of third reference ranges of 50-80 mg (PMID 32992693, meta, efficacy 1) with the weight of 30 and 75-100 mg (PMID 28515951, RCT, efficacy 1) with the weight of 1. A third weighted lower bounds of the plurality of third reference ranges can be 50*30=1500 and 75*1=75. A third weighted upper bound of the plurality of third reference ranges can be 80*30=2400 and 100*1=100. The third weighted lower bounds of the plurality of the third reference ranges 1500 and 75 can be summed together 1500+75=1575, and be divided by the summation of third weights 30+1=31 to determine a third lower dosage bound 1575/31=50.81. The third weighted upper bounds of the third reference ranges 2400 and 100 can be summed together 2400+100=2500, and be divided by the summation of third weights 30+1=31 to determine a third upper dosage bound 2500/31=80.64.

The computing system can generate a dosage vector including a dose value for each of a plurality of nutrient products (e.g., vitamin D 25 mg pill, vitamin C 10 mg pill, etc.) including the first nutrient and the one or more other nutrients. Each element in the dosage vector can be number of nutrient products that the user is recommended. For example, if the plurality of nutrient products comprises a pill of vitamin D 25 mg, a pill of vitamin D 50 mg, and a pill of zinc 20 mg, then the dosage vector of [0, 1, 2] can mean zero pill of vitamin D 25 mg, 1 pill of vitamin D 50 mg, and 2 pills of zinc 20 mg.

A score matrix that includes a nutrient-assessment score for each nutrient of each assessment can be determined. The nutrient assessment score can be determined by using a dosage value (e.g., 25 mg, 100 mg, etc.) for the dose value for the nutrient in the dosage vector relative to a dosage bound for the nutrient. Each row can represent each assessment, and each column can represent different nutrients used in the assessments. The nutrient assessment score can be determined using a following function. For each nutrient in the assessment, an assessment score can be calculated:
1) 0-1 when dosage value<lower dosage bound
2) 1 when dosage value>=lower dosage bound and dose value<=upper dosage bound
3) 0-1 when dosage value>upper dosage bound
   where the dosage value is a dosage value (e.g., 100 mg of vitamin D) of the dose value (e.g., 2 pills of vitamin D 50 mg) of each nutrient from the dosage vector, the lower dosage bound is lower dosage bound of the nutrient, and the upper dosage bound is upper dosage bound of the nutrient. The 1 can be a specified value, and depending on the amount the dosage value exceeds the dosage bound, the nutrient assessment score can decrease from the specified value.

When the dosage value is smaller than the lower dosage bound, then the following formula can be used to determine the nutrient-assessment score:

$${( \frac{lower dosage bound - dosage value}{lower bound} )}^{\frac{1}{2}}$$

where the dosage value is a dosage value (e.g., 100 mg of vitamin D) of the dose value (e.g., 2 pills of vitamin D 50 mg) of the nutrient and the lower dosage bound is lower dosage bound of the nutrient. The nutrient-assessment score may have a value between 0 and 1.

When the dosage value is greater than the upper dosage bound, then the following formula can be used to determine the nutrient-assessment score:

$${( \frac{dosage value - upper dosage bound}{tolerable upper limit - upper bound} )}^{2}$$

where the dosage value is a dosage value (e.g., 100 mg of vitamin D) of the dose value (e.g., 2 pills of vitamin D 50 mg) of the nutrient and the upper dosage bound is upper dosage bound of the nutrient. The tolerable upper limit can be the highest level of nutrient intake that is likely to pose no risk of adverse health effects for almost all individuals in the general population. The tolerable upper limit can be determined by National Institutes of Health (NIH). When the dosage value of the nutrient exceeds the tolerable upper limit, then the nutrient can be excluded from the possible recommendation (i.e., have the nutrient-assessment score can be 0). The nutrient-assessment score may have a value between 0 and 1.

For example, a score matrix for the first assessment of vitamin D deficiency and the second assessment of Goal Immunity can be determined. The first column can be an assessment score for vitamin D and the second column can be an assessment score for zinc. The first row can be the first assessment, and the second row can be the second assessment. The first assessment of vitamin D deficiency can have the dosage range of 33.33 mg and 58.33 mg for vitamin D. The dosage vector of [0, 1, 2] for a pill of vitamin D 25 mg, a pill of vitamin D 50 mg, and a pill of zinc 20 mg has in total 50 mg of vitamin D and 20 mg of zinc. The row values of the first assessment can be [1.0, 0], as the dosage value 50 mg of vitamin D is between 33.33 mg and 58.33 mg, and the first assessment does not have a recommendation for zinc. The second assessment of Goal immunity can have the dosage range of 55 mg and 222.22 for vitamin D. Since the dosage value 50 mg is smaller than 55 mg, the assessment score for vitamin D of the second assessment can be 0.8 (a value lower than 1). The second assessment of Goal Immunity can have the dosage range of 50.81 to 80.64 for zinc. Sine the dosage value 100 mg is over the upper limit of zinc 80.64, the assessment score for zinc of the second assessment can be 0.7 (a value lower than 1). Therefore, the score matrix for the dosage vector of [0,1,2] for the first assessment of vitamin D deficiency and the second assessment of Goal Immunity can be [[1.0,0],[0.8,0.7]].

Upon determining the score matrix, an assessment score can be determined for each assessment. The assessment score for each assessment can be determined using the score matrix and the assessment's maximum efficacy. Using the maximum efficacy, different scores in score matrix can be
1) if 3, the highest score is considered per assessment
2) If 1 or 2, an average of the two highest scores per assessment can be taken
3) If 0.5, an average of the three highest scores per assessment can be taken
   The averages can be weighed using the same scientific evidence weights. This can give a single assessment score per assessment. For example, the first assessment of vitamin D deficiency can have a maximum efficacy of 3 and the second assessment of Goal Immunity can have the maximum efficacy of 2. Therefore, for the score matrix of [[1.0,0],[0.8,0.7]], the first assessment score for the first assessment can be 1.0, as 1.0 is the highest score in the assessment, and the second assessment score for the second assessment can be (0.8+0.7)/2=0.92.

Upon determining the assessment scores, different assessment weights depending on severity, or particular deficiency for the user, can be determined. The severity can reflect the magnitude of the detriment to health if not treated (i.e., how severe or life threatening is this particular assessment). For example, for the first assessment of vitamin D deficiency and the second assessment of goal immunity, the first assessment of vitamin D deficiency can have worse severity than the goal immunity. Therefore, the assessment weight of 4 can be given for the first assessment of vitamin D deficiency while the assessment weight of 2 can be given for the second assessment of goal immunity. In some embodiments, the assessment weights can additionally be determined by other priorities such as the extent to how effective supplementation (including nutrient and doses delivered) can help with the member's assessment. For example, an assessment which is hard to measure and may take a very long time to correct (i.e. goal-immunity, or high cholesterol) may have a lower business priority score than an assessment we can readily influence given our formulary (i.e. low vitamin D with a higher dose vitamin D regimen.)

A total score can be determined by multiplying the assessment score with the corresponding assessment weight to determine a weighted assessment score. The weighted assessment scores can be summed and divided by the summation of weights. For example, the first assessment score 1 can be multiplied by 4 and the second assessment score 0.75 can be multiplied by 2 to determine weighted assessment scores of 4 and 1.5. Then the weighted assessment scores can be summed 4+1.5=5.5 and divided by the summation of assessment weights 4+2=6 to determine the total score of 0.92. Therefore, for a dosage vector [0,1,2] of 25 mg of vitamin D, 50 mg of vitamin D, and 20 mg of zinc, the first assessment of vitamin D deficiency and the second assessment of Goal Immunity can have the total score of 0.92.

The dosage vector can be optimized by optimizing a total score. To optimize the total score, the computing system can use a machine learning technique to use different combinations of dosages in the dosage vector to determine the highest score. The dosage vector with the highest total score can then be determined as an optimal dose. For example, different combinations of dosage vectors [1,1,2], [0,2,1], etc. can be used to determine different total scores, and the dosage vector with the highest total score can then be chosen as optimal doses. The algorithm in computing the total score may be designed such that the dosage vectors with the lowest dosage amount that can hit dosage ranges of different nutrients throughout assessments can have the highest total score.

The computing system can put several constraints to make optimizing dose values in the dosage vector more efficient and practical. Example constraints can be each nutrient having an upper dosage limit (e.g., 500 mg of vitamin D), each nutrient product having upper dose value bound (e.g., having upper dose value of 3 for 50 mg vitamin D), the total dose values in the dosage vector being 7, etc.

The smart protein and the optimal dose for each of the plurality of nutrient products (e.g., supplements) can be provided to the user. A combination product, or smart protein and the nutrient products, can then be given to a user. In some embodiments, the optimal doses of the plurality of nutrient products can be part of the smart protein. The smart protein can contain protein, carbohydrate, flavor, and added nutrients.

### B. Flowchart

Methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps.

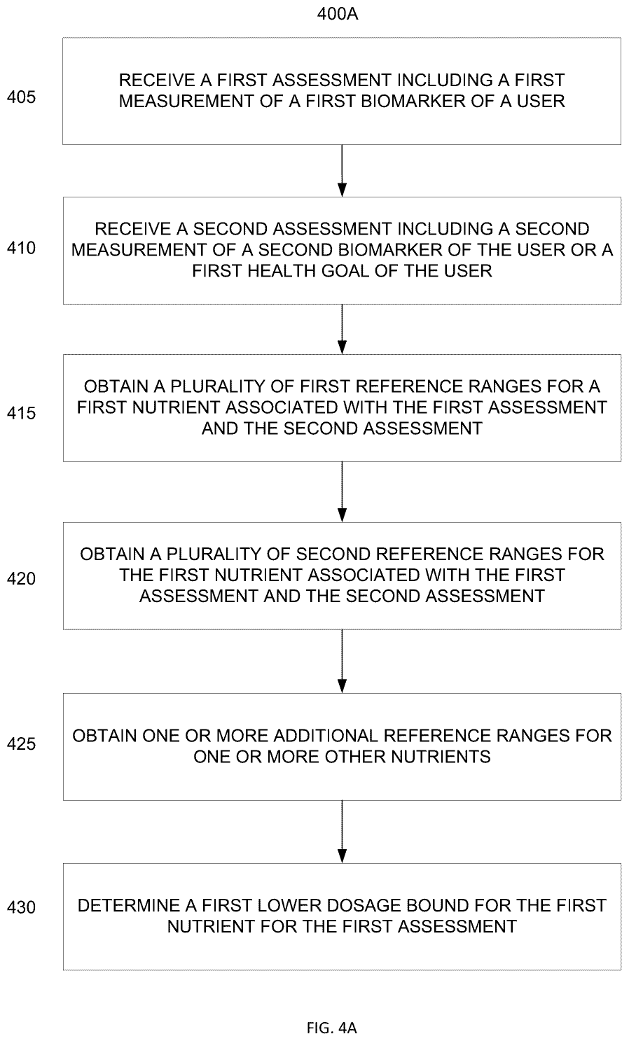

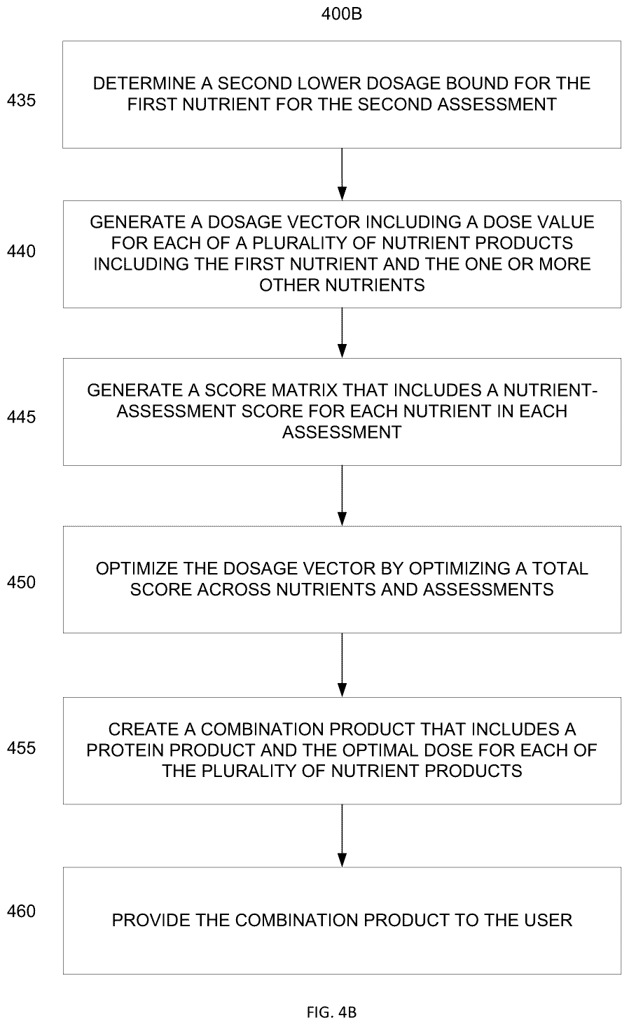

FIGS. 4A and 4B show a flow chart for determining dosing for added nutrients (i.e., micronutrients) according to some embodiments. The method 400A and 400B show a computer system receiving first assessment and second assessment to determine an optimal dose for each of a plurality of nutrient products.

In step 405, the computing system can receive a first assessment including a first measurement of a first biomarker of a user. The first assessment can be obtained from a biomarker data. The first assessment can be a nutrient deficiency found from the biomarker data. For example, the first assessment can be vitamin D deficiency if the first measurement of the vitamin D (e.g., the first biomarker) is low. The biomarker data can be obtained by the user performing a blood work.

In step 410, the computing system can receive a second assessment including a second measurement of a second biomarker of the user or a first health goal of the user. The second assessment can be obtained from the biomarker data or questionnaire data. The second assessment can be a nutrient deficiency found from the biomarker data or the first health goal of the user. For example, the second assessment can be vitamin C deficiency if the second measurement of the vitamin C (e.g., the first biomarker) is low. The second assessment can be the first health goal of the user's immunity.

The computing system can query a database using the first assessment and the second assessment. The database can comprise pluralities of reference ranges for different nutrients obtained in steps 410 to 420.

In step 415, the computing system can obtain a plurality of first reference ranges for a first nutrient associated with the first assessment and the second assessment. The plurality of first reference ranges can be obtained by using different sources such as scientific literatures, research papers, etc. The plurality of first reference ranges can be associated with corresponding first efficacies and corresponding first weights of the first nutrient for the first assessment. The first reference ranges can be ranges of doses of the first nutrient recommended to the user to improve the condition of the first assessment. The first efficacies can be numerical value indicating effectiveness of the plurality of first reference ranges in improving the condition of the assessment based on evidence. The higher first efficacies can indicate that the associated plurality of first reference ranges have high level of reliable clinical evidence supporting them. The corresponding first weights can be determined by identifying a type of source (e.g., meta-analysis, systematic review, etc.) used to determine the associated plurality of first reference ranges. The types of sources with stronger evidence can be given higher weights.

In step 420, the computing system can obtain a plurality of second reference ranges for the first nutrient associated with the first assessment and the second assessment. The plurality of second reference ranges can be obtained by using different sources such as scientific literatures, research papers, etc. The plurality of second reference ranges can be associated with corresponding second efficacies and a corresponding second weights of the first nutrient for the second assessment. The second reference ranges can be ranges of doses of the first nutrient recommended to the user to improve the condition of the second assessment. Similar to the first efficacies and the first weights, the second efficacies and the second weights can be determined.

In step 425, the computing system can obtain one or more additional reference ranges for one or more other nutrients. An assessment, such as a first assessment and a second assessment, can have more than one nutritional needs. For example, for the assessment of Goal immunity, it requires vitamin D and zinc to improve the condition of the assessment. In such case, pluralities of reference ranges for vitamin D and zinc can be obtained.

In step 430, the computing system can determine a first dosage bound for the first nutrient of the first assessment using the plurality of first reference ranges and the corresponding first weights of the first nutrient for the first assessment. A first lower dosage bound of the first dosage bound can be determined using lower bounds of the plurality of the first reference ranges and the corresponding first weights. A first upper dosage bound of the first dosage bound can be determined using upper bounds of the plurality of the first reference ranges and the corresponding first weights. The first lower dosage bound and the first upper dosage bound can then be used to identify the first dosage bound.

In step 435, the computing system can determine a second dosage bound for the first nutrient of the second assessment using the plurality of second reference ranges and the corresponding second weights of the first nutrient for the second assessment. A second lower dosage bound of the second dosage bound can be determined using lower bounds of the plurality of the second reference ranges and the corresponding second weights. A second upper dosage bound of the second dosage bound can be determined using upper bounds of the plurality of the second reference ranges and the corresponding second weights. The second lower dosage bound and the second upper dosage bound can then be used to identify the second dosage bound.

In step 440, the computing system can generate a dosage vector including a dose value for each of a plurality of nutrient products including the first nutrient and the one or more other nutrients. Examples of nutrient products can be pills such as 10 mg of vitamin D, 50 mg of vitamin D, 10 mg of vitamin C, etc. Each element in the dosage vector can indicate the dose value for the each of the plurality of nutrient products. For example, the dosage vector of [0,1,2] for the plurality of nutrient products comprising 10 mg of vitamin D, 50 mg of vitamin D, and 10 mg of vitamin C can mean 1 pill of 50 mg of vitamin D and 2 pills of 10 mg of vitamin C. The total doses for each nutrient can be 50 mg of vitamin D and 20 mg of vitamin C.

In step 445, the computing system can generate a score matrix that includes a nutrient-assessment score for each nutrient for each assessment. The nutrient-assessment score can be dependent on the dose value for the nutrient relative to a dosage bound for the nutrient for each assessment. Upon determining the score matrix, an assessment score for each assessment can be determined using the nutrient-assessment scores for nutrients in the assessment and the efficacies for corresponding nutrients for the plurality of reference ranges. The assessment score for each assessment can then use assessment weights to determine a total score across nutrients and assessments.

In step 450, the computing system can optimize the dosage vector by optimizing the total score across nutrients and assessments. The computing system can optimize the dosage vector by using a machine learning technique to try different doses of the dosage vector to find the highest total score. The dosages of the dosage vector with the highest total score, or the optimal dose for each of the plurality of nutrient products, can then be obtained.

In step 455, the computing system can create a combination product that includes a protein product and the optimal dose for each of the plurality of nutrient products. The protein product can be smart protein mentioned in FIG. 2. The smart protein can comprise protein, carbohydrates, and flavor. In some embodiments the combination product can have the optimal doses of the plurality of nutrient products in the smart protein. In step 460, the computing system can provide the combination product to the user.

### V. Smart Protein Packaging

Once different doses of nutrients in smart protein are determined using data (e.g., blood biomarker, activity data, questionnaire), the smart protein can be packaged. The package can have different information that can help a user identify different ingredients, amount of scoops for a workout, etc.

FIG. 5 shows smart protein packaging 500 according to some embodiments. The smart protein packaging can comprise ingredients list 502 and a QR code 504. The smart protein packaging can comprise other information such as mission statement 506 and brand name 508.

The ingredients list 502 can comprise different ingredients that were used to make the smart protein 200. The ingredients can inform the user of different food materials that the smart protein is made of In some embodiments, the ingredients can show different percentage of the ingredients used. For example, the ingredient can show the percentage of protein to carbohydrates to indicate what activity score (e.g., no carb, low carb, med carb, high carb) the smart protein has. The percentage of different ingredients used in carbohydrate (e.g., oat starch, dextrin, etc.) can also be shown in the main ingredients. The ingredients list 502 can list different ingredients that were used in protein 202, carbohydrates 204, added nutrients 206, and flavor 208 of the smart protein 200.

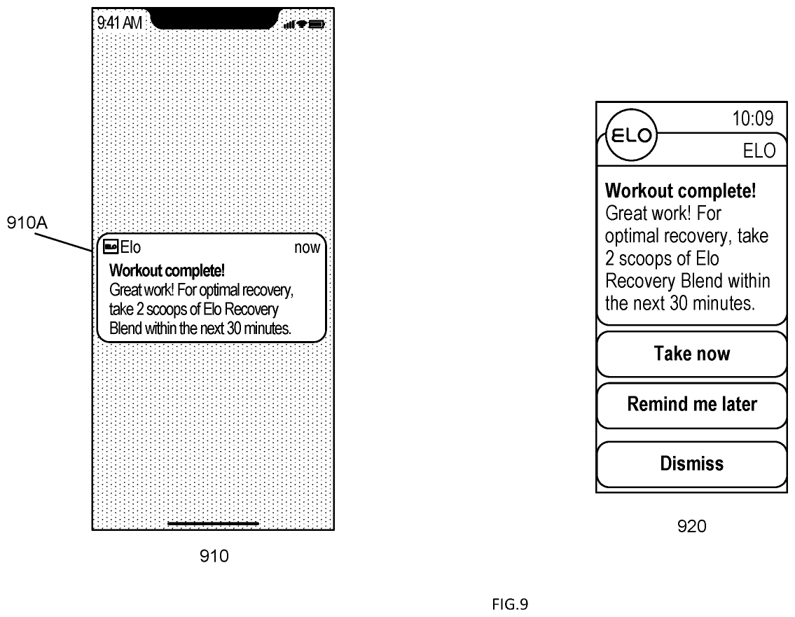

The QR code 504 can link to the user's individual product page in the user's application or web browser. The individual product page can comprise detailed explanations of different ingredients of the smart protein 200, user activity data, personalized dosing, etc. By using the user activity data in the individual product page, the embodiment can determine activity-based doses of the smart protein 200 for the user. Detailed information of the individual product page can be found in FIG. 9. Detailed information of determining activity-based doses can be found in FIG. 6.

### VI. On-Demand Dosing

Upon determining different doses of nutrients personalized for a user to make smart protein, different activity-based doses can be determined for the user depending on the type of activity. For example, the user would not have the same doses of smart protein after running for an hour than doing Yoga for an hour, as running is an activity that led to more calorie burn. An algorithm can be used to determine an ideal activity-based dose for an activity.

### A. Determining On-Demand Dosing

One or more user devices can be used to collect movement data of the user. The one or more user devices can measure current movement data, which is movement data representative of the activity the user is currently engaged on. The collection of measuring different current movement data over a specified number of days can lead to historical movement data. Measuring the current movement data can involve measuring user data including height, weight, gender, and age, the user's heartbeat, duration of the activity, type of the activity, etc. The user device, in addition to measuring the user's current movement data, can collect other health information as well (e.g., heartbeat). The user's current movement data and other health information can be collected using an application.

FIG. 6 shows an exemplary mobile page of movement data (i.e., activity) according to some embodiments. A page 610 can show detailed explanations of an exemplary high intensity interval training activity. The page 610 can comprise activity description 612, progress bar 614, and checkmark 616. The activity description 612 can comprise activity-based doses 612A, current active calorie burn 612B, total active calorie burn 612C, total time 612D, and average heart rate 612E.

The doses (e.g., activity-based doses 612A, reference dose etc.) recommended to a user can be simplified in half-increments for user ease. For example, instead of recommending 1.7 scoops, 1.5 or 2 scoops can be recommended to a user. The following algorithm can be used to simplify doses recommendation.

| | |
|---|---|
| a. | Anchored to the reference dose in FIG. 2, rounded down to the nearest half-scoop. |

| | |
|---|---|
| i. | Example. A reference dose of 1.3 scoops would yield an anchor dose of 1 |
| | scoop. |

| | |
|---|---|
| b. | Upper-bounded by 3 half-scoop increments from the anchor (e.g., reference) dose. |

| | |
|---|---|
| i. | Example. The maximal recommendation for someone who has a 1 scoop |
| | anchor dose would be 1 → 1.5 → 2 scoops. |

| | |
|---|---|
| c. | Bottom-bounded by a minimum of 1 scoop, with the addition of half-scoop |
| | increments up to the anchor dose above. A few example sets of possible activity- |
| | based Smart Protein recommendations include: |

| | |
|---|---|
| i. | Example. |

| | |
|---|---|
| 1. | Anchor dose = 1 scoop. |
| 2. | Set of possible recommendations = 1, 1.5, 2. |

| | |
|---|---|
| d. | The reference (and subsequent anchor) doses depend upon product formulation |
| | (addition of carbohydrates and protein type inform how much leucine is delivered |
| | per dose) and achieved activity level. |
| e. | Otherwise, for each activity, the dose should be regularly rounded to the nearest |
| | half-scoop. The formulate for activity-adjusted dosing is below. |

| | |
|---|---|
| i. | Example. If the activity-adjusted dose is 1.3 scoops, the final |
| | recommendation should be 1.5 scoops. |

The activity-based dose for an activity (e.g., movement) can be determined by using a historical active calorie burn rate, an active calorie burn for the activity, and a reference dose. The reference dose can be determined by performing calculations done in FIG. 2. The historical active calorie burn rate can be determined using historical movement data representative of activity levels of the user over a specified number of days received from the one or more user devices. The historical movement data can then be used to determine historical active calorie burn, which can be divided with the specified number of days to determine a historical active calorie burn rate. The active calorie burn for the activity can be determined by using a current movement data representative of the activity of the user, the current movement data received within a specified time window of the activity occurring (e.g., start and end of the activity). The current movement data can also be measured using one or more user devices. The activity-based dose can be determined by using the following formula:

Activity based dose=Reference dose*(Activity Calorie Burn/Historical active calorie burn rate)

An example of determining activity-based doses can be shown below. The example can be a continuation of previous example from FIG. 2, where the reference dose is 1.3 scoops, has average active calorie burn per day (i.e., historical active calorie burn rate) is 534 calories per day. Different active calorie burn for different activities can be shown in the example. Additionally, in cases where the activity-based dose is less than 0.5 scoops, “No Protein Needed” recommendation can be generated.
Reference dose=1.3 Scoops
Set of possible recommendations: 1, 1.5, and 2 scoops
Average Active Calorie Burn/Day (Last 30 Days): 534 calories/day
Recent Workout Active Calorie Burn: 708, 568, 348, 135
Ex 1: Activity based dose=1.3*(708/534)=1.8 scoops=2 scoops
Ex 2: Activity based dose=1.3*(568/534)=1.4 scoops=1.5 scoop
Ex 3: Activity based dose=1.3*(348/534)=0.85 scoops=1 scoop
Ex 4: Activity based dose=1.3*(135/534)=0.3 scoops=No Protein Needed

By using the algorithm to simplify the doses, with the reference dose being 1.3 scoops, the reference dose is underestimated to 1 scoop, and three half scoop increments of 1, 1.5, and 2 scoops can be recommended. Using the activity-based dose formula, an initial value for the activity-based dose can be determined. For example, the initial value can be 1.8 scoops, 1.4 scoops, etc. from the example. One of a plurality of predetermined doses that corresponds to the initial value can then be selected as the activity-based dose. The predetermined doses can be initial doses rounded to half increments according to the algorithm.

The activity description 612 can comprise the activity-based doses 612A, the current active calorie burn 612B, the total active calorie burn 612C, the total time 612D, and the average heart rate 612E. The activity-based doses 612A can doses of smart protein that is recommended to the user to take after the activity. The activity-based doses 612A can be determined by using the reference dose, the current activity calorie burn 612B, and the historical active calorie burn. The current active calorie burn 612B can be the calorie burn from doing the current activity. The current active calorie burn 612B can be determined from using the current movement data received from one or more user devices. The total active calorie burn 612C can be total calories that were spent by the user. This can include the activity and other things (e.g., breathing) that can require calorie use. The total time 612D can be the time frame of the current activity.

The progress bar 614 can indicate current status of reaching the historical active calorie burn. The status 614A can be the current active calorie burn, the status 614B can be the historical active calorie burn. Looking at the example status in FIG. 6, it can be shown that the user needs to do more activity to reach the historical active calorie burn for the day.

The user can decide to complete the activity and decide to take the activity-based dose of smart protein. The user can make a record of taking the activity-based dose of the smart protein by clicking the checkmark 616. The user can perform other activities to hit the mark of historical active calorie burn, in which the embodiment can then calculate the activity-based dose according to the other activities.

### B. Flowchart

Methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps.

FIG. 7 shows a flow chart for determining an activity-based dose of smart protein. The method 700 shows a computer system receiving movement data including historical movement data and current movement data of a current activity to use the movement data to determine the activity-based dose a user can take after the current activity.

In step 710, the computing system can receive historical movement data from one or more user devices of a user. The historical movement data can be representative of activity levels of the user over a specified number of days (e.g., 30 days). For example, the movement data can track different activities such as running, playing basketball, yoga, etc. that the user did over the specified number of days. The historical movement data can be measured by the one or more user devices (e.g., Smart Watch). The movement data can be tracked and stored by the one or more user devices by using an application. Different activity details such as time, heart rate, amount of distance traveled, etc. can be measured by the one or more user devices. These activities can be collected over the specified number of days to determine the movement data.

In step 720, the computing system can determine a historical active calorie burn rate. The historical active calorie burn rate can be determined using historical movement data representative of activity levels of the user over the specified number of days received from the one or more user devices. The historical movement data can then be used to determine historical active calorie burn, which can be divided with the specified number of days to determine a historical active calorie burn rate. For example, if 40000 active calories have burned in the past 40 days through different activities measured by one or more user devices, then the average calorie burn rate can be determined as 1000 calories per day.

In step 730, the computing system can receive current movement data from one or more user devices of a user. The current movement data can be representative of current activity of the user. The current movement data can be received within a specified time window of the activity occurring. The specified time window can be a time period in which the activity-based dose should be taken. The current movement data can be measured by the one or more user devices. The collection of current movement data from over the specified number of days of the past can result in the historical movement data. The current movement data can be stored using the application. Different activity details such as time, heart rate, amount of distance traveled, etc. can be measured by the one or more user devices.

In step 740, the computing system can receive a current active calorie burn using the movement data. The current active calorie burn can be calorie burn of the for the current activity the user is performing. Determining the current active calorie burn can be dependent on different factors: the intensity of the activity and user data such as gender, weight, etc. For example, running leads to higher calorie burn than yoga as running leads to more movements. The computing system can take into account all these factors when determining the current active calorie burn. In some embodiments, the one or more user devices can determine the calorie burn of the current activity. For example, in FIG. 6, the user device can measure the current movement data of the user and determine the current active calorie burn. The one or more user devices can also measure other health data such as heartbeat.

In step 750, the computing system can determine an activity-based dose of a smart protein comprising a protein dosage based on the historical active calorie burn rate and the current active calorie burn. The activity-based dose can first determine reference dose by matching with 3 grams of leucine amino acid described in FIG. 2. The reference dose can then be used along with the historical active calorie burn rate and the current active calorie burn to determine an initial value for the activity-based dose of the smart protein through the formula where the activity-based dose is equal to the reference dose multiplied by the current calorie burn divided by the historical active calorie burn rate. The initial value can then be rounded to the nearest half dose according to the estimation technique described in FIG. 6.

In step 760, the computing system can send a notification to a user device to the user. The notification can include the activity-based dose for the user to ingest within the specified time window. An example of the notification can be shown in FIG. 9. The notification can be generated by using the application that measures the current movement data and the historical movement data. The application can then determine the historical active calorie burn rate and the current active calorie burn, use the formula to determine the activity-based dose, and send the notification to the user device such that the user can be reminded to take the smart protein.

### VII. Collection of Movement Data

One or more user devices can be used to measure movement data (e.g., real-time activity data) to determine ideal carbohydrate dosage for the smart protein. The real-time activity data can be detected and transmitted within a specified time constraint, e.g., seconds, minutes or hours. The real-time activity data can also be used to determine ideal activity-based doses, or smart protein doses a user takes after different activities. The one or more user devices can identify the ideal post-workout recovery window based on each recorded activity (i.e., movement data). By using the real time activity data from the one or more user devices, embodiment can give a recommendation of activity-based doses the user can take after the activity. Historical active movement data, or a collection of real time activity data over a period of time, can be used to determine the ideal carbohydrate dosage of the smart protein. Examples of such user devices that can measure movement data include: a watch, a phone, a bicycle, glasses, a wristband, treadmill, elliptical machine (or any other cardio machine), and the like. Such measurements can be performed using an inertial motion sensor, such as an accelerometer and/or a gyrometer.

Embodiments can have an individual product page in the user's application or web browser that comprise detailed information on smart protein and the movement data. The detailed information can comprise data on the ingredients used to make smart protein, nutrients in the smart protein, history of movement data, current movement data, recommendation of activity-based doses, etc. The activity-based doses can be determined by using the current movement data and the history of movement data received from the one or more user devices.

FIG. 8 shows exemplary mobile pages for activity history and ingredients of smart protein according to some embodiments in a mobile device. A page 810 can comprise the user's movement and health record. The page 810 can comprise health map 812, smart supplement status 814, and activity history 816. A page 820 can comprise detailed information about the smart protein. The page 820 can comprise smart protein general information 822, activity list 824, and ingredient list 826.

The page 810 can comprise user's health and activity record. The health map 812 can comprise a health status of different nutrients in the user's body when it first started out with the plan. In some embodiments, the health map 812 can show most recent health status of the user. The health map 812 can show the status of nutrients such as hemoglobin, glucose, cholesterol, C-reactive protein (CRP), vitamin D, creatine, etc. The user health map 812 can be obtained by using biomarker data. The smart supplement status 814 can track the frequency in which the user correctly consumed the smart protein each day. For example, for each day the user correctly consumes the smart protein, a green bar can be filled. The details of the user intake of the smart protein can further be seen by clicking show details option 814A. The activity history 816 can show average daily activity energy 816A, forecasted activity level 816B, and activities done by the user such as strength training 815C and outdoor walk 815D.

The average daily activity energy 816A can be determined by using historical movement data representative of activity levels of the user over a specified number of days (e.g., 30 days) received from the one or more user devices. The historical movement data can then be used to determine historical active calorie burn, which can then be divided with the specified number of days to determine a historical active calorie burn rate. For example, if 30000 active calories have burned in the past 30 days, then the average calorie burn rate can be determined as 300 calories per day. Measuring the historical movement data can involve measuring user data including height, weight, gender, and age, the user's heartbeat, durations and types of different activities over the specified number of days. The forecasted activity level 816B can be determined by tracking whether the user's active calorie burn currently is on track to reach the historical active calorie burn rate. For example, if the user's active calorie burn currently for the day is 300 calories the historical active calorie burn rate is 350 calories/day, then since the user is on track to hit the total active calorie burn rate of the smart protein, the forecasted activity level can show it to be moderate.

A list of activities the user performed such as strength training activity 815C and outdoor walk 815D can be shown in the activity history 816 in the order in which they've been completed (e.g., most recent activities show on top). Each activity can show the active calorie burn for the activity, time the activity was completed, and the activity-based dose the user is recommended to take for the activity. The details to how the activity-based dose is determined can be found later. By clicking the activity, the mobile device can lead to a page similar to page 800 of FIG. 8 comprising detailed information about the activity. Other activities not shown in the activity history 816 can be shown by clicking an option 816E to show all

A page 820 can comprise detailed information about the smart protein. The smart protein general information 822 can comprise a general description of the smart protein, size of the package, flavor, existence of carbohydrate, status, etc. The smart protein general information 822 can additionally comprise shipment information 822A of next smart protein for next time period (e.g., month), and a manage option 822B that can enable the user to change necessary changes (e.g., ingredients, activity score, etc.) to the smart protein the user desires. The activity options in the activity list 824 can be similar to activity options 816D and 816E provided in the page 810. The details to how the number of scoops, or doses, can be calculated can be shown to the user by clicking the option 824A (which will be explained in FIG. 6). The ingredient list 826 can show detailed explanations of which ingredients are used and the clinical reason for which they've been included. For example, ingredient 826A show detailed description of whey protein and the clinical reason behind why the whey protein was included.

Current movement data can be received from the one or more user devices. The current movement data can represent an activity of the user and have a specified time window during the activity. For example, the user can perform Yoga and the one or more user devices can be used to measure movement data from the start to finish (e.g., time frame) of the Yoga.

At the end of the activity, the user can be notified with a notification of the completed activity. The notification can be received through one or more user devices. The notification can be timed to alert the user of its real-time ideal recovery windows, during which smart protein should be consumed to prevent increased muscle soreness. An example of the notification can be shown in FIG. 9. A mobile device in page 910, where a notification 910A can show the end of the activity with an instruction including the recovery window and the activity-based dosing recommendation. Another example of the notification can show in a smart watch in page 920. The page 920, similar to page 910, can comprise a notification with an instruction similar to the notification 910A.

### VIII. Computer System

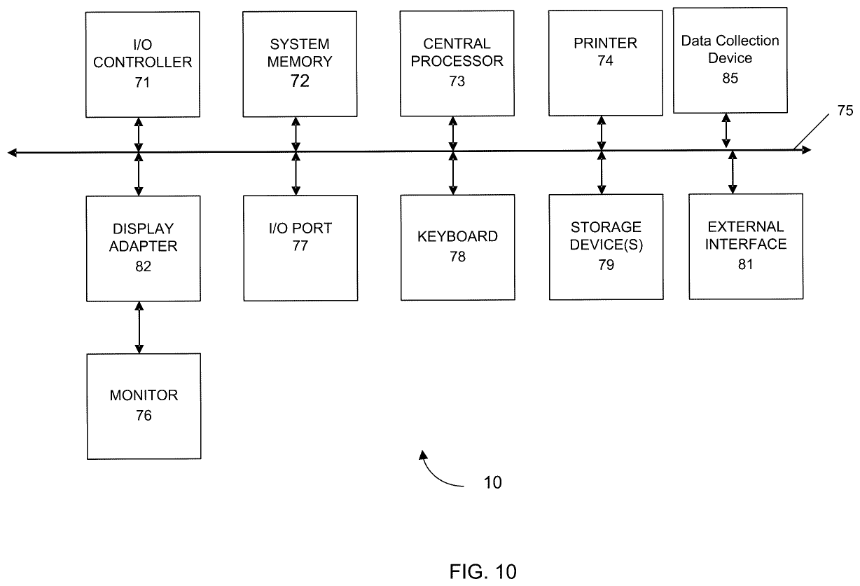

Any of the computer systems mentioned herein may utilize any suitable number of subsystems. Examples of such subsystems are shown in FIG. 10 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 10 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire©). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor may be performed in real-time. The term “real-time” may refer to computing operations or processes that are completed within a certain time constraint. The time constraint may be 1 second, 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

What is claimed is:

1. A method comprising:
receiving, by a computing system, movement data from one or more user devices of a user, the movement data being representative of activity levels of the user over a specified number of days, wherein the movement data is measured by the one or more user devices;
receiving, by the computing system, user data including height, weight, age, and gender;
determining, by the computing system, a total active calorie burn using the movement data;
determining, by the computing system, an activity score using the total active calorie burn and the user data;
determining, by the computing system, a carbohydrate dose based on the activity score; and
mixing the carbohydrate dose with a protein dose to obtain a protein-carbohydrate composition for the user.

2. The method of claim 1, wherein determining the activity score using the user data includes:
determining a user's basal metabolic rate (BMR) using the user data; and
determining the activity score includes using the user's BMR.

3. The method of claim 1, wherein determining the activity score includes using a user preference rate.

4. The method of claim 1, wherein determining the carbohydrate dose based on the activity score includes:
determining a quantity ratio between a protein and a carbohydrate using the activity score; and
determining the carbohydrate dose using the quantity ratio.

5. The method of claim 1, wherein the carbohydrate dose specifies a quantity of each of a plurality of carbohydrates.

6. The method of claim 1, wherein the movement data includes at least one of: a user's heartbeat measured during different activities over the specified number of days, a type of one or more activities performed over the specified number of days, and an amount of distance traveled by the user over the specified number of days.

7. The method of claim 1, wherein mixing the carbohydrate dose with the protein dose comprises:
adding the carbohydrate dose to a mixer machine;
adding the protein dose to the mixer machine; and
mixing the carbohydrate dose and the protein dose in the mixer machine until homogeneity is achieved to obtain the protein-carbohydrate composition.

8. A method comprising performing, by a computing system:
receiving historical movement data from one or more user devices of a user, the historical movement data being representative of activity levels of the user over a specified number of days, wherein the historical movement data is measured by the one or more user devices;
determining a historical active calorie burn rate using the historical movement data;
receiving current movement data from the one or more user devices of the user, the current movement data being representative of an activity of the user, the current movement data received within a specified time window of the activity occurring, wherein the current movement data is measured by the one or more user devices;
determining a current active calorie burn using the current movement data;
determining an activity-based dose of a nutrient composition comprising a protein dosage based on the historical active calorie burn rate and the current active calorie burn; and
responsive to receiving the current movement data, sending a notification to a user device to the user, the notification including the activity-based dose for the user to ingest within the specified time window.

9. The method of claim 8, wherein determining the historical active calorie burn rate using the historical movement data comprises:
determining an historical active calorie burn using the historical movement data; and
determining the historical active calorie burn rate includes using the historical active calorie burn and the specified number of days.

10. The method of claim 8, wherein determining the activity-based dose comprises:
determining a reference dose using a body weight dose, a maximum protein dose, and a minimum protein dose; and
determining the activity-based dose includes using the reference dose.

11. The method of claim 8, wherein determining the activity-based dose includes:
determining an initial value for the activity-based dose; and
selecting one of a plurality of predetermined doses that corresponds to the initial value as the activity-based dose.

12. A method comprising:
receiving a first assessment including a first measurement of a first biomarker of a user;
receiving a second assessment including a second measurement of a second biomarker of the user or a first health goal of the user;
querying a database using the first assessment and the second assessment to:
obtain a plurality of first reference ranges for a first nutrient associated with the first assessment, wherein the plurality of first reference ranges is associated with corresponding first efficacies and corresponding first weights of the first nutrient for the first assessment;
obtain a plurality of second reference ranges for the first nutrient associated with the second assessment, wherein the plurality of second reference ranges is associated with corresponding second efficacies and corresponding second weights of the first nutrient for the second assessment; and
obtain one or more additional references ranges for one or more other nutrients;
determining a first dosage bound for the first nutrient of the first assessment using the plurality of first reference ranges and the corresponding first weights of the first nutrient for the first assessment;
determining a second dosage bound for the first nutrient of the second assessment using the plurality of second reference ranges and the corresponding second weights of the first nutrient for the second assessment;
generating a dosage vector including a dose value for each of a plurality of nutrient products including the first nutrient and the one or more other nutrients;
generating a score matrix that includes a nutrient-assessment score for each nutrient for each assessment, wherein the nutrient-assessment score is dependent on the dose value for the nutrient relative to a dosage bound for the nutrient;
optimizing the dosage vector by optimizing a total score across nutrients and assessments, thereby obtaining an optimal dose for each of the plurality of nutrient products, wherein the total score is determined using the score matrix and an efficacy for the nutrient for each reference range for each assessment;
creating a combination product that includes a protein product and the optimal dose for each of the plurality of nutrient products; and
providing the combination product to the user.

13. The method of claim 12, further comprising:
determining one or more nutrient deficiencies associated with the first assessment; and
identifying the first nutrient associated with the first assessment using the one or more nutrient deficiencies associated with the first assessment.

14. The method of claim 12, further comprising:
determining one or more nutrient deficiencies associated with the second assessment; and
identifying the first nutrient associated with the second assessment using the one or more nutrient deficiencies associated with the second assessment.

15. The method of claim 12, wherein the plurality of first reference ranges is associated with corresponding first types of sources, wherein the corresponding first types of sources are used to determine the corresponding first weights.

16. The method of claim 12, wherein the plurality of second reference ranges is associated with corresponding second types of sources, wherein the corresponding second types of sources are used to determine the corresponding second weights.

17. The method of claim 12, wherein the nutrient-assessment score is smaller than a specified value when a dosage value of the dose value exceeds the dosage bound.

18. The method of claim 17, wherein the nutrient-assessment score decreases from the specified value based on an amount the dosage value of the dose value exceeds the dosage bound.

19. The method of claim 12, wherein determining the total score includes using an assessment score and assessment weight for each assessment, wherein the assessment score is determined using the score matrix and the efficacy for the nutrient for each reference range for each assessment, and wherein the assessment weight is determined based on severity of nutrient deficiencies or health goals of the user.

20. The method of claim 12, wherein determining the first dosage bound comprises determining a first lower dosage bound and a first upper dosage bound.

21. The method of claim 20, wherein determining the first lower dosage bound includes:
multiplying each lower bound in the plurality of first reference ranges with the corresponding first weights to determine first weighted lower bounds; and
determining the first lower dosage bound by dividing a summation of the first weighted lower bounds by a summation of the corresponding first weights.

22. The method of claim 20, wherein determining the first upper dosage bound includes:
multiplying each upper bound in the plurality of first reference ranges with the corresponding first weights to determine first weighted upper bounds; and
determining the first upper dosage bound by dividing a summation of the first weighted upper bounds by a summation of the corresponding first weights.
